SEQUENCE LISTING (4 sequences)

SEQ ID NO: 1 - DNA, 21 bp, Artificial
  forward primer
cacaggcatatgactagtggc

SEQ ID NO: 2 - DNA, 18 bp, Artificial
  reverse primer
ggaggtggcgctcgcagc

SEQ ID NO: 3 - DNA, 30 bp, Artificial
  forward primer
acgcgtcgacagaaactatgtgctgtagta

SEQ ID NO: 4 - DNA, 29 bp, Artificial
  reverse primer
cggaattccctattaaaaggttaaaacgt

(19) United States
(12) Patent Application Publication
(10) Pub. No.: US 2008/0034455 A1
(43) Pub. Date: Feb. 7, 2008

(54) Production Of A Protein Localized To The Endoplasmic Reticulum Protein Bodies In A Transgenic Plant And Seed

(75) Inventors: Allison R. Kermode, West Vancouver (CA); Xu He, Burnaby (CA); Lorne Clarke, West Vancouver (CA)

Correspondence Address: WILMERHALE/NEW YORK, 399 PARK AVENUE, NEW YORK, NY, 10022, US

(73) Assignee: SIMON FRASER UNIVERSITY, Burnaby, BC (CA)

(21) Appl. No.: 11/596,633

(22) PCT Filed: May 19, 2005
(86) PCT No.: PCT/CA05/00762

§ 371 Date: Oct. 11, 2007

Related U.S. Application Data
(60) Provisional application No. 60/573,777, filed on May 21, 2004.
(60) Provisional application No. 60/598,428, filed on Aug. 4, 2004.

(51) Int. Cl. A01H 5/00; C07H 21/04; C12P 21/06

(52) U.S. Cl. 800/298; 435/69.1; 536/23.6

(57) ABSTRACT

A nucleotide sequence is provided for expression of a heterologous protein of interest that is localized to the endoplasmic reticulum protein bodies in transgenic plants, plant cells, and seeds. Said nucleotide sequence comprises a promoter, the 5′ untranslated region from the maize γ-zein gene, the γ-zein signal peptide sequence, a sequence encoding a heterologous protein of interest, and the 3′ untranslated region from the maize γ-zein gene, operatively linked.

### FIELD OF INVENTION

The present invention relates to production of proteins in plants. More specifically this invention pertains to the production of glycoproteins in plants.

### BACKGROUND OF THE INVENTION

Lysosomal storage diseases are a broad class of human genetic disorders involving deficiencies of specific hydrolytic enzymes that reside within lysosomes (Scott et al., 1995). The mucopolysaccharidoses (MPS) are a group of 11 disorders resulting from deficiencies of enzymes involved in glycosaminoglycan degradation. MPS I (Hurler's syndrome), the most frequent of the generalized MPS disorders, results from a deficiency of α-L-iduronidase. This disorder is associated with pathologic effects in most tissue types and is thus considered to represent the prototypical MPS disease. While generalized pathology occurs, it is the skeletal, cardiac and neurological disturbances that are most severe and lead to death in early childhood.

Lysosomal diseases are amenable to enzyme therapies (ERT or Enzyme Replacement Therapy) and, to date, the FDA has approved the use of three lysosomal enzymes (produced as recombinant proteins) for treatment: α-L-iduronidase (for MPS I), glucocerebrosidase (for Gaucher's disease) and α-galactosidase (for Fabry disease). Six other recombinant enzymes are at the Phase III or proclinical stages. However, the current methods used to produce these enzymes (most commonly human fibroblasts or Chinese hampster ovary cells) are very expensive. For example, ERT using recombinant human α-iduronidase produced by Chinese hampster ovary cell cultures is estimated at about US $500,000 per patient per year; for Gaucher disease, annual costs for a typical 60 kg patient are approximately US $85,000 to US $350,000, depending on dosage.

Transgenic plants or plant cells are potentially one of the most economical systems for large-scale production of recombinant proteins for industrial and pharmaceutical uses (Obermeyer et al., 2004; Twyman et al., 2003; Ma et al. 2003; Daniell et al. 2001). Plant expression systems have advantages over other systems as production costs are relatively low and plants cells are not susceptible to contamination by human pathogens as can occur in mammalian expression systems. Human collagens, human growth hormones and antibodies have been produced in plants and these plant-derived proteins appear to have biological activities similar to those of the native proteins (e.g. Dieryck et al., 1997; Ma and Hein 1995). For example, recombinant antibodies produced in tobacco plants have the same sensitivity, specificity, and importantly, the same affinity as monoclonal antibodies produced by the original hybridoma cell line.

When produced in a transgenic plant system, human or animal recombinant proteins synthesized with a native signal peptide are destined for transport within the plant secretory system. With no additional topogenic sequences, secretion at the cell surface is the most likely outcome after transit of the recombinant protein through the ER and Golgi complex (see Kermode 1996). The different glycosylation patterns mediated by Golgi-traversed enzymes in plant cells are a major limitation to utilizing plants as biofactories for production of animal or human therapeutic proteins, the majority of which require specific glycosylation to attain their native forms.

Plants and mammals share the first steps of N-linked glycosylation that take place co-translationally within the ER. In this process, an ER glucosyltransferase transfers a group of sugar residues (Glc₃Man₉GlcNAc₂) en bloc from a lipid dolichol intermediate onto the N atom of an Asn residue of the nascent polypeptide chain. Only those proteins with the consensus sequence Asn-X-Ser/Thr are subject to N-linked glycosylation.

Plant and animal cells also subject the original N-linked glycan on the protein to “trimming”, a process in which the 3 terminal glucose residues (and generally one mannose residue) are sequentially removed while the protein is still in the ER lumen. It is only following transit of the recombinant protein to the next compartment of the secretory pathway (the Golgi complex) that problems arise due to differences in the nature of Golgi-localized enzymes of plant versus animal cells.

Within the Golgi complex of both plant and animal cells, enzymes convert the original high mannose glycans of proteins to complex glycans, a process that involves a series of sequential reactions and relies on accessibility of the glycan chain(s) to the Golgi processing machinery. The reactions involved in converting high mannose glycans to complex glycans in both plant and animal cells include:

(1) trimming of mannose residues;

(2) addition of N-acetylglucosamine residues and

(3) the addition of new sugars.

However, details of the process of complex glycan formation that differ in plant and animal cells. In mammalian cells, the Man₃GlcNAc₂ (M3) core structure is extended further to contain penultimate galactose and terminal sialic acid residue (Kornfeld and Kornfeld, 1985). In contrast, typically processed N-glycans of plant proteins are mostly of a Man₃GlcNAc₂ structure with or without β,2-xylose or a 1,3-fucose residues (e.g. Rayon et al., 1999).

N-linked glycans of human lactoferrin produced in maize endosperm are mostly of the paucimannose-type (Man₃GlcNAc₂-based structures). The complex glycans of plants are generally much smaller than those of mammals and, when larger, contain additional a1,4-xylose and p1,3-galactose residues, giving rise to mammalian Lewis^{a} (LE) structures (Lerouge et al., 1998). The presence of xylose, fucose, or both xylose and fructose residues makes plant recombinant therapeutics less desirable (Chrispeels and Faye, 1996; Lerouge et al., 1998) because they are potentially highly immunogenic (Bakker et al., 2001).

There have been several attempts to manipulate the glycosylation status of plant-derived human and animal proteins. For example, an Arabidopsis thaliana mutant defective in the gene encoding N-acetylglucosaminyltransferase I (a Golgi enzyme) does not generate proteins with xylose- and fucose-containing complex glycans (Von Schaewen et al., 1993) and thus has potential for generating recombinant animal or human glycoproteins with high mannose N-linked glycans. N-acetylglucosaminyltransferase I initiates the conversion of high-mannose N-linked glycans to complex N-linked glycans in plant as well as in animal cells (Gomez and Chrispeels, 1994). In the absence of this activity, the xylosyl- and fucosyl-transferases of the plant Golgi do not add xylose and fucose respectively, leaving the glycan in a trimmed, but non-complex, form (Manₛ_{_{—}}₆GlcNac₂; Lodish et al., 2000).

Other attempts to alter glycosylation pathways in plant cells have focused on the expression of mammalian enzymes in plants (e.g. human β1,4-galactosyltransferase, the first glycosyltransferase of mammalian cells that initiates the further branching of complex N-linked glycans after the action of N-acetylglucosaminyltransferases I and II; Palacpac et al., 1999). Tobacco BY suspension cells engineered in this manner generate glycoproteins that possess glycans that react with Ricinus communis agglutinin 120 (specific for β1,4-linked galactose), do not react with an antibody specific for complex glycans containing β1,2-xylose residues and have no detectable a1,3-fucose residues (determined by HPLC and IS-MS/MS).

Within animal cells, soluble lysosomal proteins are glycosylated in the ER and, upon arrival in the cis-Golgi, modifications by specific Golgi enzymes (N-acetylglucosamine phosphotransferase and phosphoglucosidase) ultimately result in mannose-6-phosphate (M6P) tags on the protein (Kornfeld and Mellman, 1989). The specificity of the reaction lies with the enzyme N-acetylglucosamine phosphotransferase, which recognizes a conformation-specific signal patch on lysosomal hydrolases, such that only proteins destined to reside in lysosomes are appropriately modified. Later in the pathway, the M6P tag allows the proteins to bind to M6P receptors and hence be specifically sorted for delivery to the lysosome in the trans-Golgi network. Release of lysosomal proteins from their receptors occurs in an acidified (pre-lysosomal, late endosomal) compartment; subsequently, the receptors recycle back to the Golgi complex (or to the cell surface because these receptors are also involved in endocytosis) while the proteins are packaged into lysosomes.

Plant cells do not contain lysosomes. The acidic plant cell vacuole is involved in macromolecular degradation and thus is often referred to as the “lysosomal equivalent”. However, plant vacuolar proteins, which transit through the ER and Golgi complex are not modified to contain M6P tags and N-linked glycans are not involved in vacuolar targeting (Kermode, 1996).

Enzyme replacement therapies use purified recombinant human lysosomal enzymes delivered to the patient intravenously. This requires that the enzyme is competent for entry into human cells and for intracellular transport to the lysosome. There are two pathways involved in uptake: an M6P-independent pathway for uptake into macrophages (requiring high mannose glycans on the hydrolase) and an M6P-dependent pathway required for uptake into most other cells. Because it is possible to subsequently generate M6P tags in vitro, it may be appropriate to generate plant-derived recombinant human lysosomal enzymes containing high mannose glycans.

U.S. Pat. No. 5,929,304 (Radin et. al., which is incorporated herein by reference) disclose the production of human protein, including IDUA in tobacco. The expression constructs comprises sequences encoding a signal peptide (SP) that either targets the protein to the plant vacuole (proaleurain SP), out of the cell (PR-1 SP), or into the ER using a KDEL sequence.

U.S. Pat. No. 5,270,200 (Samuel et. al.) teaches the synthesis of a seed storage protein from Phaseolus vulgaris, arcelin, that is toxic to pests.

WO 01/129242 (Bassuner et. al.) provides methods for post-translationally modifying heterologous polypeptides produced in a plant involving expressing one or more post-translational modification enzymes in the host plant.

Cereal grains such as rice and maize accumulate a major class of storage proteins (prolamins) in ER-derived protein bodies. Prolamins do not transit through the Golgi complex when accumulating in the ER-derived protein bodies (Larkins et al., 1989; Herman and Larkins, 1999). Without wishing to be bound by theory, in rice, signals on the prolamin mRNA molecules may be involved in targeting to specific subdomains of the ER membrane, avoiding sequestration of proteins encoded by the prolamin mRNA into vesicles enroute to the Golgi complex. More specifically the prolamin mRNAs are targeted to protein-body ER instead of cistemal ER (Choi et al, 2000). In maize, retention of zeins in ER-derived protein bodies may occur via a similar mechanism involving mRNA targeting, or ER retention may be mediated by the tertiary structure of zein proteins (Coleman, et al., 1996; Baggar et al., 1997).

### SUMMARY OF THE INVENTION

The present invention relates to production of proteins in plants. More specifically this invention pertains to the production of glycoproteins in plants.

It is an object of the invention to provide an improved protein production system in plants.

According to the present invention there is provided a nucleotide sequence comprising:

a first nucleic acid sequence encoding a signal peptide that localizes a protein of interest fused to the signal peptide to an endoplasmic reticulum (ER)-derived protein body within a cell;

a second nucleic acid sequence encoding the protein of interest; and

a regulatory element operatively linked with the first nucleotide sequence,

wherein one or more than one of the first nucleic acid, the second nucleic acid and the regulatory element, is heterologous with respect to one or more than one of first nucleic acid, the second nucleic acid and the regulatory element. Preferably, the protein body is a cereal grain protein body

The present invention pertains to the nucleotide sequence described above wherein the signal peptide is a prolamins signal peptide. Furthermore, the prolamins signal peptide may be γ-zein signal peptide. The nucleic acid may further comprise a γ-zein 3′UTR and a γ-zein 5′UTR.

The present invention also related to the nucleotide sequence described above wherein the regulatory element is selected from the group consisting of a constitutive regulatory element, an inducible regulatory element, and a tissue specific regulatory element. The regulatory element may be a γ-zein regulatory element.

The present invention also provides a plant, a plant cell, or a seed comprising the nucleotide sequence as described above.

The present invention also provides a nucleotide sequence comprising, a γ-zein regulatory element operatively linked with a nucleic acid sequence encoding a γ-zein signal peptide fused to a heterologous protein of interest, and a γ-zein 3′UTR operatively linked to the nucleic acid sequence. The present invention also includes a plant, a plant cell, or a seed comprising the nucleotide sequence as just described.

The present invention provides an expression cassette or a vector comprising a nucleotide sequence comprising:

a first nucleic acid sequence encoding a signal peptide that localizes a protein of interest fused to the signal peptide to an endoplasmic reticulum (ER)-derived protein body within a cell;

a second nucleic acid sequence encoding the protein of interest; and

a regulatory element operatively linked with the first nucleotide sequence,

wherein one or more than one of the first nucleic acid, the second nucleic acid and the regulatory element, is heterologous with respect to one or more than one of first nucleic acid, the second nucleic acid and the regulatory element.

The present invention also provides a method of producing a protein of interest within a plant comprising,
i) providing the plant comprising a nucleotide sequence a nucleotide sequence comprising:

a first nucleic acid sequence encoding a signal peptide that localizes a protein of interest fused to the signal peptide to an endoplasmic reticulum (ER)-derived protein body within a cell;

a second nucleic acid sequence encoding the protein of interest; and

a regulatory element operatively linked with the first nucleotide sequence,

wherein one or more than one of the first nucleic acid, the second nucleic acid and the regulatory element, is heterologous with respect to one or more than one of first nucleic acid, the second nucleic acid and the regulatory element; and

ii) expressing the protein of interest.

The present invention pertains to the method defined above, wherein in the step of expressing (step ii), the protein of interest is expressed in cereal grain. Furthermore, the protein of interest may be isolated from the cereal grain, and the protein of interest may be purified.

The present invention also provides an expression construct comprising the following operatively linked elements:
P-γ5′-UTR-γSP—X-γ3′-UTR,
where:

P is a seed-specific, tissue specific, or constitutive promoter;

γ5′-UTR is the 5′ untranslated region from the maize γ zein gene, or a portion thereof;

and γ3′-UTR is the 3′ untranslated region from the maize γ zein gene, or a portion thereof;

γSP is a nucleic acid encoding a signal peptide obtained from the maize γ zein gene, or a portion thereof; and

X is a nucleotide sequence encoding protein of interest fused to the γSP

The present invention also provides a plant, a plant cell, or a seed comprising the nucleotide sequence as described above.

The present invention provides a method to express human or animal proteins in plants where the recombinant proteins are targeted to endoplasmic reticulum (ER)-derived protein bodies. The protein bodies may be within cereal grains. By targeting protein to the ER-derived protein bodies, transit to the Golgi complex is avoided and synthesis of recombinant proteins containing high mannose glycans in plant cells can be produced. This method avoids maturation of protein N-linked glycans that involve the addition of potentially immunogenic sugar residues such as xylose, fucose or both xylose and fucose.

This invention also relates to the manipulation of glycosylation of recombinant human and animal proteins of interest produced in plant expression systems. The expression construct for synthesis of the recombinant protein of interest uses the nucleotide sequence encoding the mature animal or human protein flanked by regulatory sequences, for example the promoter, 5′ untranslated region, signal peptide and 3′ untranslated region of maize zein genes. These sequences are used to direct a recombinant protein of interest to ER-derived protein bodies via a Golgi-independent pathway by exploiting the plant host cells mRNA targeting machinery.

The invention provides a means of producing animal or human proteins with high mannose N-linked glycans, thus avoiding the generation of plant-like complex glycans that are potentially immunogenic. For example, the production of human lysosomal enzyme α-L-iduronidase in transgenic maize seeds demonstrates that using the construct of the present invention targets the protein of interest to endoplasmic reticulum-derived protein bodies.

Zein storage proteins, or prolamins, of maize seeds are deposited in ER-derived protein bodies and do not transit through the Golgi complex. In rice, mRNA targeting to the protein body-ER facilitates prolamin localization in ER-derived protein bodies.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows constructs for expression of human lysosomal enzyme α-L-iduronidase (IDUA). FIG. 1a shows bd35S-AMV-IDUA-nos; FIG. 1b shows bd35S-AMV-SP-IDUA-3′UTR. Constructs (a) and (b) were used for transient expression in maize calli (see Examples); construct (b) was also used for stable expression of IDUA in maize calli. db35S, enhanced 35S promoter; AMV, alfalfa mosaic virus leader sequence; nos, nopaline synthase polyA addition signal; SP, γ-zein signal peptide; 3′-UTR, 3′untranslated region of γ-zein gene.

FIG. 2 shows transient expression of IDUA in maize calli. Protein was extracted from maize calli that were bombarded with: lane 1, gold particles without DNA; lane 2, db35S-AMV-zein SP-IDUA-3′UTR; lane 3, db35S-AMV-IDUA-Nos. FIG. 2a shows IDUA activities of transformants. FIG. 2b shows western blot analysis of transformants using an antibody specific for human IDUA.

FIG. 3 shows constructs used for expression of IDUA. FIG. 3a shows a control contruct comprising an IDUA signal peptide and nos terminator. FIG. 3b shows a construct comprising a γ-zein signal peptide and 3′ UTR (also referred to as “test” or “targeting” construct). Arrow donates 5′ upstream sequence including promoter and 5′ untranslated region. SP: signal peptide; UTR: untranslated region.

FIG. 4 shows IDUA expression in transgenic maize seeds. FIG. 4a shows. RT-PCR to detect IDUA transcripts in developing endosperm of maize. UT, untransformed control; 1-3, independent transgenic lines; pc, positive control; M, DNA marker. Arrow indicates the expected size of the amplified fragment of IDUA. FIG. 4b shows western blot analysis to detect IDUA protein level in transgenic maize endosperm of T1 mature seeds. 1-17, independent transgenic lines.

FIG. 5 shows northern and western blot analysis of IDUA RNA and protein, respectively, in developing endosperm of transgenic maize T1 seeds. FIG. 5a: Northern blot to detect α-L-iduronidase (IDUA) transcripts in the transgenic developing maize endosperms. The ˜2.2 kb IDUA transcripts were detected in different transgenic lines (top panel 1-8); ribosomal RNAs were stained by ethidium bromide to verify the quantity of RNA loaded (lower panel). UT, untransformed control; lanes 1-7, independent transgenic lines expressing test construct; lane 8, transgenic line expressing control construct (see FIG. 3a). FIG. 5b: western blot analysis to detect α-L-iduronidase in transgenic maize seeds. Crude proteins were extracted from endosperms of T1 developing seeds expressing the test construct (see FIG. 3b). UT, untransformed control, 1-6, indicate independent transgenic lines. Equal protein (100 μg) was loaded in each lane. M=pre-stained molecular weight markers. Arrow indicates the expected size of the IDUA protein. Some of the immunoreactive lower molecular mass bands on the westerns of the transgenic lines may represent proteolytically cleaved α-L-iduronidase.

FIG. 6 shows immunolocalization of human α-L-iduronidase (IDUA) in developing maize endosperm cells. FIG. 6a: Confocal images of fluorescent-immunolabeled sections of maize endosperm. γ-Zein and IDUA were labeled with Cy2- or Cy3-conjugated secondary antibodies. Arrowheads indicate protein bodies. Bar=10 μm. FIG. 6b: Transmission electron microscope images of gold-immunolabeled ultrathin sections of maize endosperm. γ-Zein and IDUA were labeled with 5-nm (arrowheads) and 10-nm (arrows) gold-conjugated secondary antibodies, respectively. Shown are protein bodies in maize endosperm cells expressing the test construct (left; see FIG. 3b for construct) or control construct (right; see FIG. 3a for construct). The inset on the upper left corner shows the two enlarged sizes of gold. Bar=100 nm.

FIG. 7 shows localization of human α-L-iduronidase by western blot analysis of subcellular fractions generated by discontinuous sucrose gradients. Homogenates of maize endosperms from transgenic lines expressing the test construct (FIG. 7a; see FIG. 3b for construct) and the control construct (FIG. 7b; see FIG. 3a for construct) were separated on discontinuous sucrose gradients, and the interfaces between layers of sucrose concentrations were collected. Ten μg protein from each interface was loaded in each lane. An exception is in FIG. 7b, lane 1, in which the supernatant of the 10% sucrose fraction was loaded. The western blot of the different fractions in FIG. 7a (right) using the anti-γ-zein and anti-IDUA antibodies shows that γ-zein and IDUA, respectively, are primarily in the protein body fraction (50-70%). The control construct shows no IDUA within the protein body fraction. M=pre-stained molecular weight markers; 10-20%, 20-50% and 50-70% indicate the sucrose concentration of the discontinuous sucrose gradients.

FIG. 8 shows endoglycosidase H digestion of the maize-expressed human α-L-iduronidase. Iduronidase was affinity-purified from transgenic maize endosperm cells expressing the “targeting” construct (left; see FIG. 3b for construct) and the “control” construct (right; see FIG. 3a for construct) and then incubated in buffer with and without Endo H (+ and −, respectively). The products were separated on a 10% SDS-PAGE gel and then immunoblotted with anti-α-L-iduronidase (polyclonal) antibodies. The migration rate of iduronidase derived from the control construct is different that derived from the “test” construct after Endo H treatment.

### DETAILED DESCRIPTION

The present invention relates to production of proteins in plants. More specifically this invention pertains to the production of glycoproteins in plants.

The following description is of a preferred embodiment.

This invention relates to the production of recombinant human and animal proteins of interest, for example but not limited to lysosomal proteins, hormone peptides, vaccines, nutritional supplements, antibodies, anticoagulants, growth factors, enzymes and the like, in a plant system. Preferably the protein of interest is targeted to endoplasmic reticulum-derived protein bodies.

The present invention provides a strategy for the plant-based synthesis of pharmaceutical and other recombinant proteins, in which the resultant protein contains only high mannose N-linked glycans. Maturation of the recombinant proteins' N-linked glycans to complex forms is avoided since there is no transit of the protein through the plant Golgi complex. This control of glycosylation is important in the utility of plant-derived therapeutics, as the addition of xylose, fucose, or xylose and fructor sugar residues have been shown to elicit immunogenic responses in mammals and to greatly reduce the efficacy of plant-derived recombinant proteins for pharmaceutical or other uses (e.g. Bardor, M. et al., Glycobiology 13, 427-434; 2003).

The use of mRNA localization signals as a strategy for ER retention of plant-synthesized recombinant proteins is also particularly attractive for downstream processing, since it does not require the fusion of mature-protein-coding sequences onto the recombinant protein. Typically additional amino acids derived from targeting domains will lead to a loss of functionality of the plant-derived recombinant protein. Moreover any production mechanism that incorporates the addition of a proteolytic cleavage site for removal of non-native amino acids is not completely effective since it generally does not remove all of the added amino acids.

Therefore, the present invention provides a nucleotide sequence comprising:

a first nucleic acid sequence encoding a signal peptide that localizes a protein of interest fused to the signal peptide, to an endoplasmic reticulum (ER)-derived protein body within a cell;

a second nucleic acid sequence encoding the protein of interest; and

a regulatory element operatively linked with the first nucleotide sequence.

It is preferred that one or more than one of the first nucleic acid, the second nucleic acid and the regulatory element, is heterologous with respect to one or more than one of first nucleic acid, the second nucleic acid and the regulatory element. The nucleotide sequence as just defined may be introduced into a plant using methods as known in the art. Furthermore, the nucleotide sequence may be inserted within a vector or cassette and used for transforming a plant.

An expression cassette for synthesis of a recombinant protein of interest is also provided. The expression cassette may comprise a nucleotide sequence encoding the protein of interest, for example a mature animal or human protein of interest, that is operatively linked to appropriate regulatory sequences, for example, a promoter, and a polyadenylation region, for example, the 3′ untranslated region of the maize γ-zein gene. Additional sequences encoding a signal peptide that is fused to the protein of interest, and a 5′ untranslated region may also be included in the expression vector. These sequences direct a recombinant protein of interest to ER-derived protein bodies via a Golgi-independent pathway by exploiting the plant host cells' mRNA targeting machinery.

An example of the construct, which is not to be considered limiting in any manner may be represented as:
P-γ5′-UTR-γSP—X-γ3′-UTR,
where:

P is a seed-specific or tissue specific, or constitutive promoter;

γ5′-UTR and γ3′-UTR are the 5′ and 3′untranslated regions, respectively, from the maize γ zein gene, or a portion thereof;

γSP is a nucleic acid encoding a signal peptide obtained from the maize γ zein gene, or a portion of nucleotide sequence that encode mature zein proteins and which facilitate the targeting of the recombinant protein to ER-derived protein bodies. Also included are nucleotide sequences from other prolamine genes that function as signals to target the protein to ER-derived protein bodies; and

X is a nucleotide sequence encoding protein of interest, for example, a lysosomal enzyme or other human or animal protein to be expressed in plant cells. However, the recombinant protein of interest is not limited to a lysosomal enzyme, or glycoproteins. For example, protein of interest of interest may include, but is not limited to, a pharmaceutically active protein, for example growth factors, growth regulators, antibodies, antigens, their derivatives useful for immunization or vaccination and the like. Such proteins include, but are not limited to, interleukins, insulin, G-CSF, GM-CSF, HPG-CSF, M-CSF or combinations thereof, interferons, for example, interferon-α, interferon-β, interferon-τ, blood clotting factors, for example, Factor VIII, Factor IX, or tPA or combinations thereof. A protein of interest may also encode an industrial enzyme, protein supplement, nutraceutical, or a value-added product for feed, food, or both feed and food use. Examples of such proteins include, but are not limited to proteases, oxidases, phytases, chitinases, invertases, lipases, cellulases, xylanases, enzymes involved in oil biosynthesis etc.

By “operatively linked” it is meant that the particular sequences interact either directly or indirectly to carry out an intended function, such as mediation or modulation of gene expression. The interaction of operatively linked sequences may, for example, be mediated by proteins that interact with the operatively linked sequences.

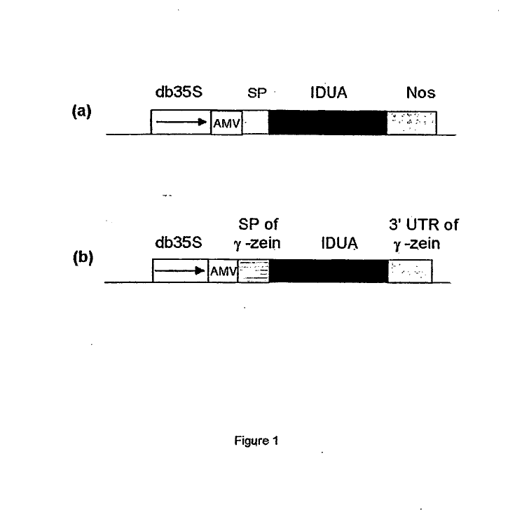

As an example, which is not to be considered limiting in any manner, a plant expression cassette for expression and synthesis of the human lysosomal enzyme α-L-iduronidase in the endosperm storage parenchyma cells of maize seeds is shown in FIG. 1b. The cassette comprises regulatory sequences from the maize γ-zein gene and includes the γ-zein promoter, the γ-zein 5′ UTR, sequences encoding the signal peptide of γ-zein (fused in-frame with sequences encoding the mature human α-L-iduronidase) and the 3′ UTR of γ-zein. However, it is to be understood that any protein of interest may be included within this cassette. Furthermore, this cassette may be used to express a protein of interest in other seeds, for example other cereal seeds, or in seeds of a plant that have been modified to express prolamine or prolamine-like proteins.

The glycosylation status and subcellular localization of the synthesized proteins derived from expression using the construct is also presented in FIG. 1. The mature human α-L-iduronidase enzyme has six consensus sequences for N-linked glycosylation (Asn-XSer/Thr; Asn 110, Asn 190, Asn 336, Asn 372, Asn 415, Asn 451 and possibly others; Zhao K W, et. al., J Biol Chem 272:22758-22765; 1997).

As described herein, inclusion of a γ-zein signal peptide significantly increases the level of a recombinant protein of interest in plant cells, and in plant seeds.

Therefore, the present invention provides a method of producing a protein of interest within a plant comprising,
i) providing the plant comprising a nucleotide sequence comprising:
   a first nucleic acid sequence encoding a signal peptide that localizes a protein of interest fused to the signal peptide to an ER-derived protein body within a cell;
   a second nucleic acid sequence encoding the protein of interest; and
   a regulatory element operatively linked with the first nucleotide sequence,
   wherein one or more than one of the first nucleic acid, the second nucleic acid and the regulatory element, is heterologous with respect to one or more than one of first nucleic acid, the second nucleic acid and the regulatory element; and
ii) expressing the protein of interest.

Preferably, in the step of expressing (step ii), the protein of interest is expressed in cereal grain. If desired, the transgenic plant comprising the protein of interest may be directly administered to an animal or human. For example, if the protein is orally administered, the plant tissue, such as the cereal grain, may be harvested and directly used, or it may be dried prior to feeding. Additionally, the harvested plant tissue may be provided as a food supplement. The protein of interest may also be extracted in either a crude, partially purified, or purified form prior to its use and isolated from the cereal grain using any suitable methods as known in the art. For example, which is not to be considered limiting, the protein of interest may be extracted using precipitation in the presence of salt or by adjusting the temperature or pH, followed by size exclusion, ion exchange or affinity chromatography.

To assist in the purification of a protein of interest, a sequence comprising an affinity epitope may be included within the protein of interest, for example but not limited to a HIS tag, or FLAG® peptide (an epitope tag; available through IBI). The protein may then be purified using a matrix that interacts with the affinity tag. The protein may also comprise a protease cleavage site, for example a thrombin cleavage sequence. By incorporating the protease cleavage site, the affinity tag portion of the protein may be removed after purification of the protein using the matrix. The matrix may comprise metal chelation or anti FLAG antibodies, or other active groups that may be used to interact with the affinity tag. However, additional amino acids derived from targeting domains may lead to a loss of functionality of the plant-derived recombinant protein. Moreover any production mechanism that incorporates the addition of a proteolytic cleavage site for removal of non-native amino acids may not be completely effective since this treatment may not remove all of the added amino acids. Therefore, using the methods as described herein, the protein of interest may be obtained in the absence of an added affinity tag or epitope.

Also provided in the present invention are stably transformed cereal plants, callus lines, and seeds that comprise the nucleic acid construct, or expression cassette, as defined herein. Examples of cereal plants include but are not limited to maize, rice, wheat and oats. Furthermore, cereal plants, cell lines and seeds provide a source of a protein of interest, for example but not limited to human α-L-iduronidase (IDUA). If desired, the IDUA can be further processed in vivo or in vitro, as would be known to one of skill in the art, to a specialized form for research or therapeutic uses.

The expression cassette as defined herein may also include, but is not limited to:

(a) DNA sequences other than the 5′ UTR, 3′ UTR and signal peptide of maize γ-zein genes. For example, coding sequences specifying part or all of the mature zein proteins which facilitate the targeting of the recombinant protein to ER-derived protein bodies may also be used;

(b) DNA sequences from other prolamin genes that function as signals to target the protein to ER-derived protein bodies may be used; and

(c) Full length or partial DNA sequences encoding mature prolamins that are in-frame with lysosomal enzymes or other proteins that can be cleaved in vivo or in vitro to produce the final proteins.

In addition, the methods described herein are not limited to expression of recombinant human and animal proteins in cereal plants and grains and, with selection of appropriate promoter sequences, either constitutive, tissue specific or inducible regulatory regions, the present method can be extended to include seeds and vegetative tissues of any other plant species including dicots. The constructs of the present invention can also include further enhancers, either translation or transcription enhancers, as may be required. These enhancer regions are well known to persons skilled in the art.

By “regulatory region” or “regulatory element” it is meant a portion of nucleic acid typically, but not always, upstream of a gene, which may be comprised of either DNA or RNA, or both DNA and RNA. A regulatory element may be capable of mediating organ specificity, or controlling developmental or temporal gene activation. A “regulatory element” includes promoter elements, basal (core) promoter elements, elements that are inducible in response to an external stimulus, elements that mediate promoter activity such as negative regulatory elements or transcriptional enhancers. “Regulatory element”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

There are several types of regulatory elements, including those that are developmentally regulated, inducible and constitutive. A regulatory element that is developmentally regulated, or controls the differential expression of a nucleotide sequence under its control, is activated within certain organs or tissues of an organ at specific times during the development of that organ or tissue. However, some regulatory elements that are developmentally regulated may preferentially be active within certain organs or tissues at specific developmental stages, they may also be active in a developmentally regulated manner, or at a basal level in other organs or tissues within the plant as well.

An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription may be present in an inactive form that is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory element may be exposed to an inducer by externally applying the inducer to the cell or plant such as by spraying, watering, heating or similar methods. Inducible elements may be derived from either plant or non-plant genes. Examples, of potential inducible promoters include, but not limited to, teracycline-inducible promoter (Gatz, C., 1997, Ann. Rev. Plant Physiol. Plant Mol. Biol. 48, 89-108; which is incorporated by reference), steroid inducible promoter (Aoyama, T. and Chua, N. H., 1997, Plant J. 2, 397-404; which is incorporated by reference) and ethanol-inducible promoter (Salter, M. G., et al, 1998, Plant Journal 16, 127-132; Caddick, M. X., et al,1998, Nature Biotech. 16, 177-180, which are incorporated by reference) cytokinin inducible IB6 and CKI1 genes (Brandstatter, I. and Kieber, J. J., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory element directs the expression of a gene throughout the various parts of a plant and continuously throughout plant development. Examples of known constitutive regulatory elements include promoters associated with the CaMV 35S transcript (Odell et al., 1985, Nature, 313: 810-812), the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646). tobacco t-CUP promoter (WO/99/67389; U.S. Pat. No. 5,824,872), the HPL promoter (WO 02/50291), and the tobacco translational initiation factor 4A gene (Mandel et al, 1995 Plant Mol. Biol. 29: 995-1004). The term “constitutive” as used herein does not necessarily indicate that a gene under control of the constitutive regulatory element is expressed at the same level in all cell types, but that the gene is expressed in a wide range of cell types even though variation in abundance is often observed.

While the invention is demonstrated by a working example demonstrating the synthesis of the human lysosomal enzyme α-L-iduronidase, the disclosed method may be used for the production of any recombinant N-linked glycan-containing protein of interest, including a human or animal protein The method described herein, including targeting and accumulation of proteins in ER-derived protein bodies of plants, can further be used as a mechanism to enhance the stability, and hence, the level of accumulation, of a protein of interest produced in plant cells regardless of its glycosylation status. Both non-glycosylated and glycosylated recombinant proteins may be produced.

Non-limiting examples of expression constructs, subcellular localization and expected glycan structures are shown in FIG. 1. In the construct shown in FIG. 1b, the human lysosomal enzyme α-L-iduronidase is operatively linked to regulatory sequences from the maize γ-zein gene.

To aid in identification of transformed plant cells, the constructs of this invention may be further manipulated to include plant selectable markers. Useful selectable markers include enzymes that provide for resistance to an antibiotic such as gentamycin, hygromycin, kanamycin, and the like. Similarly, enzymes providing for production of a compound identifiable by colour change such as GUS (glucuronidase), green fluorescence protein (GFP), or luminescence, such as luciferase are useful.

The constructs of the present invention can be introduced into plant cells using Ti plasmids, Ri plasmids, plant virus vectors, direct DNA transformation, micro-injection, bioloistics, electroporation, and other methods that would be known to those of skill in the art. For reviews of such techniques see for example Weissbach and Weissbach, (eds, Methods for Plant Molecular Biology, Academic Press Inc.; 1988), or Miki and Iyer (Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579; 1997). Methods for transformation of maize using biolositics (see for example, Fromm, et. al., 1990 Bio/Technology 8, 833-839; Gordon-Kamm, W. J., et. al., 1990, Plant Cell 2, 603-618, or stanford.edu/˜walbot/StableMaizeTransf.html; which are incorporated herein by reference), or Agrobacterium (e.g. Ishida et al., Nature Biotechnol. 14:745-750, 1996; or maizegdb.org/mnl/72/67lupotto.html; and U.S. Pat. No. 5,981,840, which are incorporated herein by reference), are known.

Plants that may be used to express the protein of interest as described herein include plants that express ER-derived protein bodies, including cereal plants for example but not limited to maize, rice, wheat and oats. Also included within the present invention are non-cereal plants that have been modified or transformed to express protein bodies comprising ER-derived or prolamin-like protein bodies. These modified plants may also be used for the expression of a protein of interest using the methods and nucleotide sequences defined herein.

Methods of regenerating whole plants from plant cells are known in the art (for example, but not limited to Armstrong, C. L. Regeneration of plants from somatic cell cultures: Applications for in vitro genetic manipulation. In The Maize Handbook; M. Freeling and V. Walbot, eds (New York: Springer-Verlag), pp. 663-671; 1994), and the method of obtaining transformed and regenerated plants is not critical to this invention. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

Avoiding the addition of immunogenic sugars such as xylose or fucose and producing recombinant protein that will be competent for appropriate intracellular (lysosomal) transport (after its in vitro modification) can be achieved by the invention described herein. A method is described that prevents transit of the recombinant protein through the plant Golgi, but allow the protein to accumulate in the ER lumen of plant cells, where N-linked glycosylation will proceed and thus generate high mannose glycans on the lysosomal protein. The predicted structure of the glycans will be: Man₅GlcNAc₂, Man₉GlcNAc₂ or both Man₅GlcNAc₂ and Man₉GlcNAc₂ depending on the degree of trimming within the ER compartment.

The present invention provides a strategy for the plant-based synthesis of pharmaceutical and other recombinant proteins. By using the nucleic acid constructs as described herein, there is no transit of the protein of interest through the plant Golgi complex, and maturation of the recombinant proteins' N-linked glycans to complex forms is avoided, and the resultant protein contains only high mannose N-linked glycans. This control of glycosylation may be advantageous in the utility of any plant-derived therapeutic as the addition of xylose and/or fucose sugar residues has been shown to elicit immunogenic responses in mammals and to greatly reduce the efficacy of plant-derived recombinant proteins for pharmaceutical or other uses (Bardor, M. et al., Glycobiology 13, 427-434; 2003).

The use of mRNA localization signals as a strategy for ER retention of plant synthesized recombinant proteins is also particularly attractive for downstream processing, since it does not require the fusion of mature-protein-coding sequences onto the recombinant protein. However, fusion proteins may also be produced according to the present invention if desired.

The present invention will be further illustrated in the following examples.

### EXAMPLES

### Example 1

### Expression of Human IDUA in Maize Calli

To investigate whether maize expressed human IDUA is enzymatically active, the following constructs were prepared using standard techniques (e.g. Maniatis et al., in Molecular Cloning (A Laboratory Manual), Cold Spring Harbor Laboratory, 1982, which is incorporated herein by reference):
double (enhanced) 35S promoter-AMV-IUDA-nos (FIG. 1a)—comprising the enhanced 35S promoter (Fang et al., Plant Cell 1, 141-150, 1989), multiple cis regulatory elements for maximal expression of the cauliflower mosaic virus 35 S promoter in transgenic plants, an AMV (alfalfa mosaic virus) leader sequence (Dalta et al., Plant Sci. 94, 139-149, 1993) the IDUA coding region (Scott, H. S., et. al., Proc Natl Acad Sci USA 88, 9695-9699; 1991; GenBank accession no. M74715), and a 3′ end derived from the Nos (Depicker et al., J Mol Appl Genet. 1, 561-573, 1982).
double 35S promoter-AMV-γ-zein SP-IUDA-γ-zein 3′UTR (FIG. 1b)—the enhanced 35S promoter, an AMV (alfalfa mosaic virus) leader sequence and the γ-zein signal peptide, the IDUA coding region;

Maize Hi-II seeds were obtained from the Maize Genetics Cooperation Stock Center (Armstrong, C. L., et. al., Maize Genet. Coop. Newsl. 65, 92-93; 1991). Maize type-II callus was initiated according to Songstad et al (Plant Cell Rep. 9, 699-702; 1991). Once the type-II callus was formed, it was transferred to maintnance medium as described by Armstrong (1994). Callus initiation and maintenance cultures were kept in the dark at 28° C. and transferred to fresh medium every 14 days. The following constructs were introduced into maize calli using particle bombardment:

1) gold particles without DNA (control);

2) db35S-AMV-zein SP-IDUA-3′UTR (FIG. 1b); and

3) db35S-AMV-IDUA-nos (FIG. 1a)

Calli were treated with 0.4 M sorbitol before and after particle bombardment.

Protein was extracted from bombarded maize calli with extraction buffer: 20 mM Tris-HCl (pH 7.5), 1 mM EDTA, 1 mM DTT, 0.5 mM PMSF, 20 μM leupeptin, 0.25 M NaCl, 1% Triton X-100. After centrifugation at 12,000 rpm for 10 min, the supernatant was removed and used for IDUA activity assays.

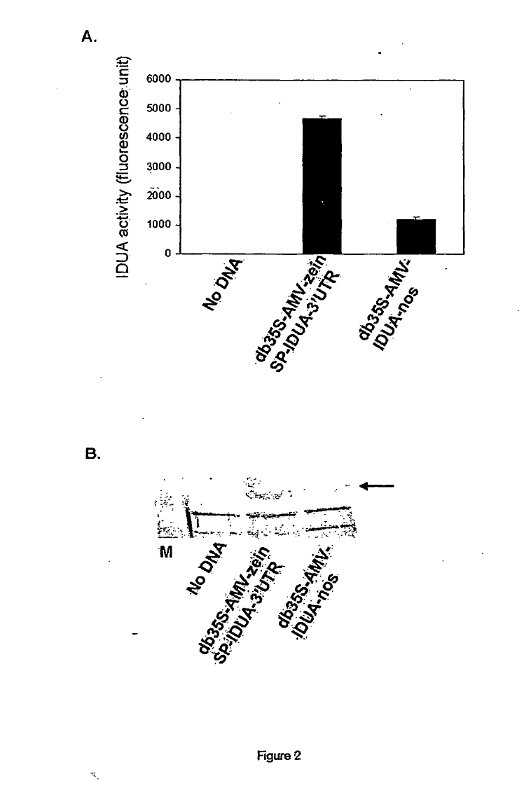

IDUA expressed in maize calli under the control of a constitutive promoter (enhanced 35S) was enzymatically active (FIG. 2a).

Further, the γ-zein signal peptide and γ-zein 3′-UTR enhanced the accumulation of IDUA as determined using Western blot analysis of transformants using an antibody specific for human IDUA (FIG. 2b).

### Example 2

### Expression of IDUA in Transgenic Maize Seeds

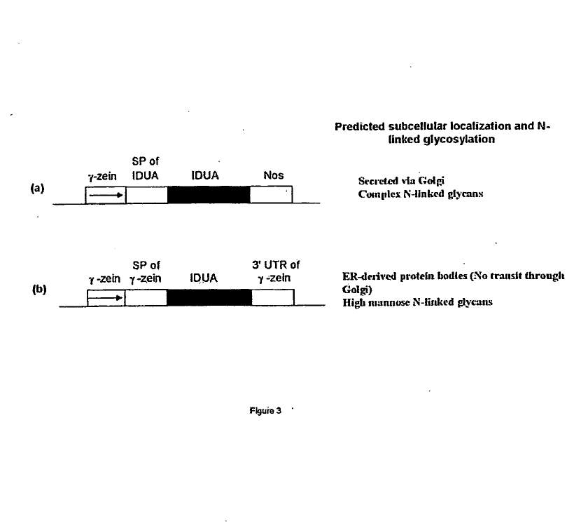

To investigate the potential of targeting the recombinant IDUA to protein bodies of maize, the human α-L-iduronidase (IDUA) gene (Scott, H. S., et. al., Proc Natl Acad Sci USA 88, 9695-9699; 1991; GenBank accession no. M74715) was driven by the 1.7-kb γ-zein promoter (obtained from the γ-zein gene; Reina, M., et. al., Nucl. Acids Res. 18, 6426; 1990; GenBank accession no. X53514). Additional regulatory sequences flanking the IDUA mature coding region included the γ-zein 5′ UTR (9 bp), the γ-zein signal peptide (57 bp) and the γ-zein 3′ UTR (191 bp) (FIG. 3b). A control construct contained the γ-zein promoter, the 5′UTR- and signal-peptide-encoding sequences of the human IDUA gene and a Nos (nopaline synthase gene) 3′ region (UTR and transcription termination sequences; FIG. 3a).

To clone the 5′ UTR, signal peptide-encoding sequences and 3′ UTR of the γ-zein gene, genomic DNA was extracted from maize Hi-II. For cloning of the 5′ UTR and signal peptide-encoding sequences, the following primers were used to PCR-amplify the fragment:

(SEQ ID NO:1)

| | |
|---|---|
| forward primer | 5′ CACAGGCATATGACTAGTGGC 3′ |
| and | |

(SEQ ID NO:2)

| | |
|---|---|
| reverse primer | 5′ GGAGGTGGCGCTCGCAGC 3′ |

For cloning of the 3′ UTR, the following primers were used:

forward primer

(SEQ ID NO:3)

5′ ACG CGT CGA CAG AAA CTA TGT GCT GTA GTA 3′
and
reverse primer

(SEQ ID NO:4)

5′ CGG AAT TCC CTA TTA AAA GGT TAA AAC GT 3′.

The DNA sequence encoding the γ-zein signal peptide was fused in-frame to the mature coding region of the IDUA gene. The final construct was verified by DNA sequencing.

The IDUA expression cassettes with (FIG. 3b) or without targeting signals (FIG. 3a) were cloned into the binary vector pCambia with Hind III and EcoRI sites and the resultant construct was transferred into Agrobacterium strain LBA4404 by electroporation. Agrobacterium-mediated transformation of maize was conducted according to U.S. Pat. No. 5,981,840, with some modifications. After the initial incubation (10 min) and 3 days of co-cultivation with LBA4404, calli were transferred to PHI-C medium [CHU(N6) basal salts (Sigma) 4.0 g/l; vitamin mix 1.0 ml/l; 2.4-D 2.0 mg/l; L-proline 0.69 g/l; sucrose 30.0 g/l; MES 0.5 g/l; phytagel 2.0 g/l; silver nitrate 0.85 mg/l; carbenicillin 100 mg/l; pH 5.8] to grow in the dark at 28° C. for 5 days. The calli were then transferred to selection media (glufosinate 3.0 mg/l in PHI-C or hygromycin 2 mg/l) and incubated in the dark at 28° C. for the first two weeks of selection. The calli were transferred to fresh selection medium at two-week intervals. After 2 months of selection, resistant calli were regenerated (Armstrong, C. L. Regeneration of plants from somatic cell cultures: Applications for in vitro genetic manipulation. In The Maize Handbook M. Freeling and V. Walbot, eds (New York: Springer-Verlag), pp. 663-671; 1994).

The Gene Encoding Human α-L-Iduronidase is Expressed in Maize Endosperm

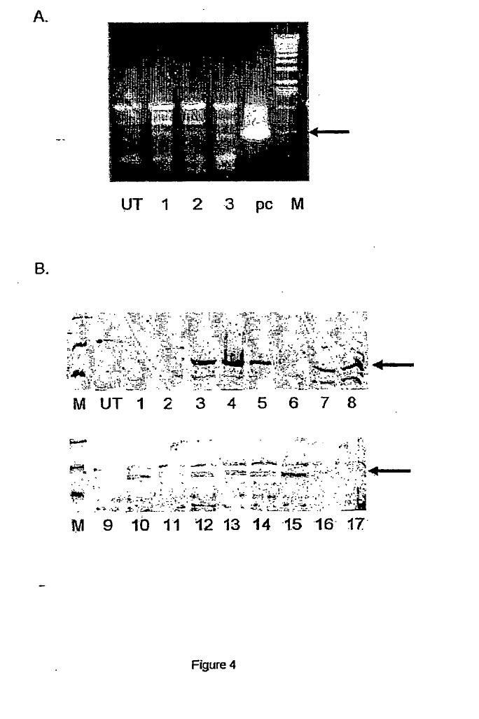

Thirty maize plants (potentially from 15 independent lines) from the control construct (see FIG. 3a) were regenerated in the presence of the selectable marker reagent and screened by PCR; 28 of the screened plants were PCR-positive for the IDUA gene. The transgenic plants were either selfed or crossed with wild-type plants to produce seeds. The developing seeds from a few independent lines were sampled and RTPCR was conducted to investigate IDUA transcription (FIG. 4a). Mature seeds were analyzed for IDUA activity and for IDUA protein using western blot analysis. IDUA protein was detected in most of the lines tested (FIG. 4b).

With the test construct (see FIG. 3b) some of the transgenic plants were sterile. However, a few lines were able to produce seeds. The T1 developing seeds were analyzed for RNA (northern blot) and protein (western blot).

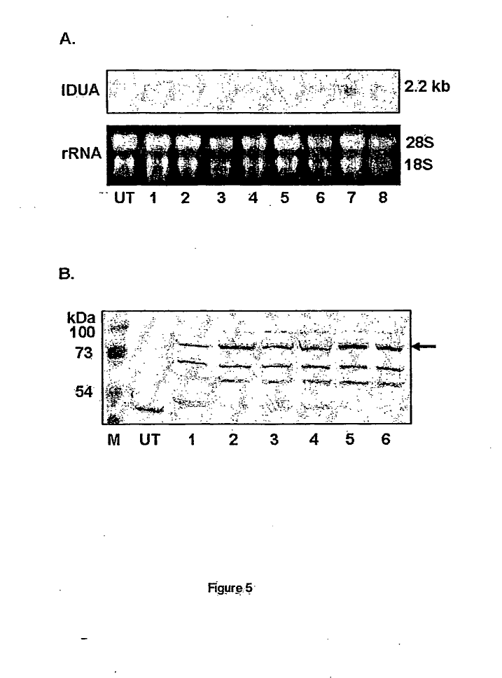

Total RNA was extracted from developing maize seeds using the Qiagen RNAeasy kit (Qiagen Inc., Mississauga, ON, Canada). Ten μg total RNA was loaded into each lane and RNA samples were fractionated on 1.0% agarose formaldehyde gels. Following transfer to Hybond™-XL nylon membrane (Amersham Life Science, Buckinghamshire, England), RNA was fixed onto the membrane by UV-crosslinking. Membranes were hybridized with a ³²P-labeled α-L-iduronidase cDNA probe; labeling was achieved using the RTS RadPrime DNA Labelling System (Life Technologies, Gaithersburg, Md., USA) using [α-³²P]-dCTP. Northern analysis using RNA extracted from developing seeds (18-20 days after pollination) of a few independent lines showed that the α-L-iduronidase gene was transcribed (FIG. 5a).

For western analysis, proteins were fractionated on 10% SDSPAGE gels and western blot analysis was carried out essentially as described in He and Kermode (Plant Mol. Biol. 52, 729-744; 2003). Anti-α-L-iduronidase and anti-γ-zein antibodies (obtained from J. Hopwood and B. Larkins, respectively) were used at dilutions of 1:100 and 1:1000, respectively. As shown in FIG. 5b, α-L-iduronidase protein, derived from expression of the test construct (FIG. 3b) in developing T1 seeds, is detected in most of the lines.

Human α-L-Iduronidase Expressed in Maize Endosperm Cells is Localized in Protein Bodies

To investigate whether α-L-iduronidase was targeted to protein bodies within maize endosperm storage parenchyma cells, immunolocalization studies were conducted on developing endosperms of transgenic seed expressing the test construct or the control construct. For this, sections were immunolabelled with antibodies against γ-zein and α-L-iduronidase sequentially.

Immunocytochemical Detection of α-L-Iduronidase and γ-Zein

Maize developing endosperms were fixed in 4% parafornaldehyde overnight at 4° C. Dehydration was performed with a graded series of ethanol: 30%, 50%, 70%, 80%, 90%, 95%, and 100%; 2 h each step. Tissues were incubated in 100% ethanol overnight at 4° C. and then taken through a graded series of LR White (Sigma Co., St. Louis, Mo., USA) in ethanol: 20%, 50%, 75%, 100%; 3 h each step. The samples were infiltrated with 100% LR White overnight and then incubated at 60° C. overnight. The samples were sectioned with an OmU3 Ultramicrotome (Reichert, Austria).

Immunolabelling of the sections was carried cut as described by Schmid et al. (Proc. Natl. Acad. Sci. USA 96, 14159-14164; 1999). Sections were then incubated in 2% BSA (in PBS containing 0.05% Tween-20) for 1 h to block nonspecific sites. The primary antibody raised against α-L-iduronidase (diluted 1:20 in blocking solution) was applied to the sections overnight at 4° C. Preimmune serum was used for the negative controls. Sections were washed three times in PBS containing 0.05% Tween-20 and incubated with a goat anti-rabbit Fab′ fragments conjugated to Cy2 (1:80 dilution in the blocking solution) for 3 h in the dark at room temperature. Fab′ fragments were used as secondary antibodies to avoid cross-reaction. After rinsing in PBS three times, sections were incubated in 2% BSA for 1 h and incubated with antibody against γ-zein (diluted 1:100 in blocking solution) for 3 h. After several washes of the sections, the sections were incubated in the secondary antibody conjugated to Cy3 (Jackson ImmunoResearch Laboratories, Inc.) for 1 h. The sections were washed 3 times and mounted in anti-FADE medium (n-propyl-gallate 6.15 mg/mL in PBS buffer containing 50% glycerol) and observed using a model LSM 410 confocal Laser Scan Microscope (excited at 488 nm for Cy2 and 568 nm for Cy3, respectively (Zeiss, Germany).

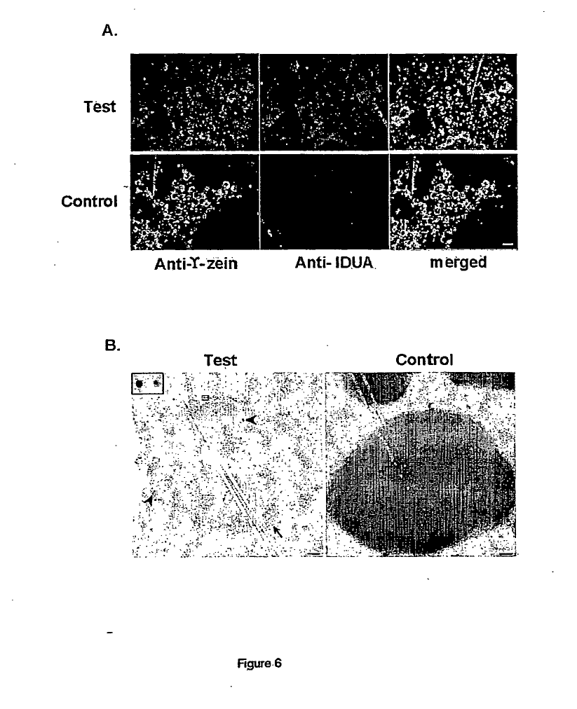

The punctuate fluorescent labeling in FIG. 6 indicates protein bodies where the γ-zein protein resides; similar fluorescent labeling indicates anti-IDUA binding within protein bodies. Co-localization of γ-zein and IDUA was determined by merging of the two micrographs and this shows that IDUA is colocalized with γ-zein protein in protein bodies. The α-L-iduronidase derived from expression of the test construct was co-localized with γ-zein protein (FIG. 6a, “Test”; construct of FIG. 3b), in contrast to that derived from expression of the control construct (FIG. 6a, “Control”; construct of FIG. 3a).

Transmission Electronic Microscopy and Immunocytochemical Detection

Developing endosperms (15-17 DAP) were fixed with a high-pressure freezer (Bal-Tec HPM 010 High Pressure Freezer, Zurich, Switzerland). Ultrathin sections were acquired using a Leica UltracutT UltraMicrotome (Reichert, Austria). The sections were picked up on 200 mesh nickel grids, and nonspecific binding sites were blocked by immersion in blocking buffer (2% normal goat serum in PBS, pH 7.2) for 1-2 h. The sections were then labeled with antibody against α-L-iduronidase (1:20 dilution) for 1 h and rinsed extensively followed by incubation with 10 nm gold-conjugated secondary antibodies (1:100 dilution) for 1 h. After rinsing several times, the sections were labeled with the antibody against γ-zein (1:50) and 5 nm gold-conjugated goat anti-rabbit Fab′ fragments (1:100), sequentially. All the antibodies were diluted in 2% goat serum blocking solution. After labeling, the grids were stained with uranyl acetate followed by lead citrate, as described by standard protocols. The grids were observed under Transmission Electron Microscopy Model Hitachi-80 (Hitachi, Tokyo, Japan).

Transmission electron microscopy further confirmed the protein body localization of the “Test” (construct shown in FIG. 3b) human α-L-iduronidase (FIG. 6b). γ-Zein protein was preferentially localized at the periphery of protein bodies (labeled by 5-nm gold particles; arrows), while α-L-iduronidase (derived from the test construct) was found both inside and at the periphery of protein bodies (labeled by 10-nm gold particles; arrowheads; FIG. 6b, left panel).

Cell Fractionation

As α-L-Iduronidase (derived from the control construct) was not readily localized using immunolocalization, cell fractionation was carried out on a discontinuous sucrose gradient.

Cell fractionation was performed according to Larkins and Hurkman (Plant Physiol. 62, 256-263; 1978). Briefly, developing maize endosperms were homogenized with buffer: 20 mM Tris-HCl (pH 7.5), 10% (w/v) sucrose, 50 mM KCl, 10 mM MgCl₂, 100 mM NaCl, 1 mM EDTA and 1 mM PMSF. After removing cellular debris by centrifugation at 500×g for 5 min at 4° C., supernatants were loaded onto three step gradients of 1 ml of 20% sucrose, 1 ml of 50% sucrose and 1 ml of 70% sucrose in the above buffer, and centrifuged at 24,000 rpm in an SW40 rotor (Beckman, Palo Alto, Calif., USA) for 2 h at 4° C. The fractions at each interface were collected and analyzed by SDS-PAGE and western blotting.

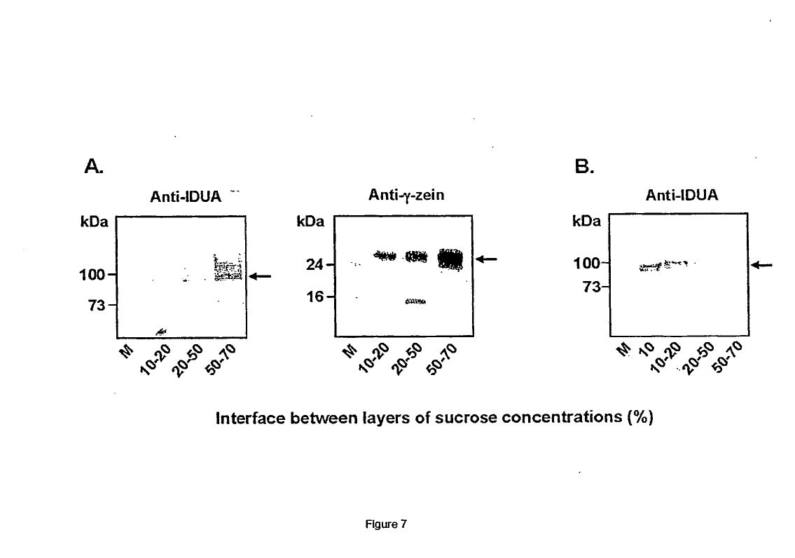

Protein bodies primarily sediment between the 50-70% sucrose layers (Larkins, B. A. & Hurkman, W. J. Plant Physiol. 62, 256-263; 1978). For the control construct (FIG. 1a), the maize-expressed α-L-iduronidase was mainly distributed in the 10%-20% portions of the sucrose gradient corresponding to soluble proteins in the cytosol and apoplast, or proteins in the microsomal fraction (FIG. 7b). In contrast, the α-L-iduronidase derived from expression of the test construct (construct of FIG. 1b: γ-zein promoter—5′UTR and signal peptide—IDUA—3′UTR) was predominantly localized in the 50-70% sucrose gradient interface corresponding to the protein body-rich fraction (FIG. 7a), similar to γ-zein.

Recombinant α-L-Iduronidase of Maize Endosperm Cells is Enzymatically Active and Contains Only High-Mannose Glycans

Despite considerable accumulation of α-L-iduronidase protein (FIG. 5b), the activity of the recombinant enzyme was influenced by several factors. For example, activities (determined by a fluorogenic assay; Clements, P. R., et. al., Eur. J. Biochem. 152, 21-28; 1985) were virtually undetectable in crude extracts, but when extracts were diluted 20-50 fold, activities increased considerably (e.g. to 70 pmol/min/mg protein; test construct).

Affinity Purification of Maize-Derived α-L-Iduronidase

Maize kernels were ground using a mortar and pestle in the presence of an equal volume of buffer A and chloroform (Buffer A: 15 mM DMG, pH 6.0, 500 mM NaCl, 1 mM CaCl2, 1 mM MnCl2, 0.01% Triton X-100, 0.01 mM DTT, 0.02% sodium azide, 0.5 mM PMSF, and 20 μM leupeptin). After centrifugation for 20 min at 3500 rpm, the supernatant was loaded onto a Con A-Sepharose column (Amersham) and eluted with buffer B (15% methyl-α-mannoside in buffer A). The eluant was concentrated with Amicon 30 kDa centrifuge filters (Millipore Corp., Bedford, Mass., USA) and loaded onto a column containing Affi-Gel-bound to monoclonal anti-α-L-iduronidase. After washing the unbound protein with buffer C (20 mM Tris, pH 7.0, 250 mM NaCl, 0.01% Triton X-100, 0.01 mM DTT, and 0.02% sodium azide), α-L-iduronidase was eluted with buffer D (50 mM sodium citrate, pH 4.0, 2 M NaCl, 0.01% Triton X-100, 0.01 mM DTT, 0.02% sodium azide) and the eluant was concentrated with Amicon 30 kDa centrifuge filters.

Following Con A-Sepharose-purification, the resultant fraction contained markedly increased specific activity (1,400 pmol/min/mg protein). Likewise, subsequent affinity purification after the Con A-Sepharose step (using Affi-Gel beads linked to a monoclonal anti-α-L-iduronidase antibody; Clements, P. R., et. al., Biochem. J. 259, 199-208; 1989) led to a further increase in specific activity (350,000 pmol/min/mg protein). Without wishing to be bound by theory, the activity of iduronidase in crude extracts of maize endosperms may be affected by endogenous inhibitors, or the human recombinant protein may refold in the presence of an immobilized matrix (Horowitz, P. M. Nat. Biotechnol. 17, 136-137; 1999).

Endo H Glucosidase Digestion and Glycan Analysis of Affinity Purified α-L-Iduronidase

In plants, complex glycans are characterized by fucose and xylose sugar residues attached to the proximal N-acetylglucosamine and core mannose residues, respectively. This modification renders the glycoprotein resistant to the Streptomyces enzyme endoglycosidase H; hence this characteristic may be used as evidence for passage of proteins through the Golgi complex. To investigate whether the N-linked glycans of the recombinant iduronidase were susceptible to Endo H cleavage, the iduronidase of crude extracts of transgenic maize endosperms was purified sequentially using Con A affinity chromatography and monoclonal anti-α-L-iduronidase chromatography, as outlined above.

Affinity-purified α-L-iduronidase was incubation in buffer containing endoglycosidase H (Endo H; New England Biolabs Inc., Mississauga, ON), or in buffer with no Endo H according to the manufacturer's instructions (37° C. 1 h) and the products were separated by SDS-PAGE. The difference in migration of the cleaved and uncleaved products was determined by western blot analysis as described above. The status of the N-linked glycans of the affinity purified α-L-iduronidase was also assessed using an anti-complex glycan antibody.

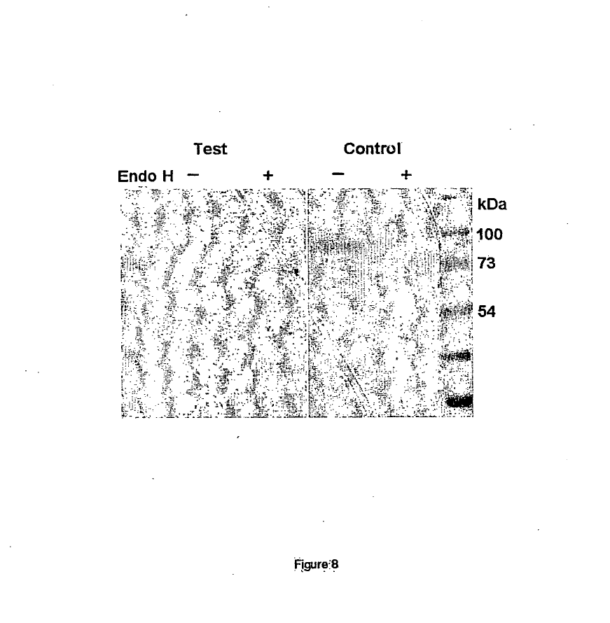

α-L-Iduronidase derived from expression of the test construct (construct of FIG. 3b) was susceptible to cleavage by Endo H, generating a product significantly lower than that of the untreated α-L-iduronidase (FIG. 8; “Test”). In addition, the protein was not recognized by an antibody against plant complex glycans (data not shown). In contrast the recombinant α-L-iduronidase generated by expression of the control construct (FIG. 3a) was also susceptible to Endo H treatment (FIG. 8, “Control”). However the cleaved product did not decline in molecular mass to the same extent as the Endo H-treated “test” iduronidase. (Although only an estimate, the difference in molecular mass between the two Endo H-treated proteins was at least 3 kDa). In addition the iduronidase, derived from expression of the control construct was recognized by the anti-complex-glycan antibodies (data not shown).

These data are consistent with the glycosylation pattern of native iduronidase. Native IDUA has six consensus signals for N-linked glycosylation in the ER; these are all utilized in human cells in which the enzyme is targeted to the lysosome via the Golgi complex. In Chinese hamster ovary (CHO) cells hosting production of the recombinant protein, the enzyme is secreted and again all six sites are used, but the N-linked glycans themselves displays high intrasite heterogeneity. Some of the N-linked glycans of the mature enzyme remain in a high mannose form (Asn 372; Asn 415 is mixed high mannose and complex); at least two of the sites are modified to complex forms (Asn 110 and Asn 190); two carry mannose-6-phosphate tags for receptor-mediated lysosomal uptake (Asn 336 and Asn 451). Thus for CHO cell-secreted recombinant iduronidase, despite transit through the Golgi complex, three or four of the six oligosaccharides are cleaved by Endo H, with both the high mannose and phosphorylated glycans remaining sensitive/susceptible to this Streptomyces glycosidase (Zhao, K. W., et. al., J. Biol. Chem. 272, 22758-22765; 1997). The combined data for the “targeted” α-L-iduronidase, for example, greater Endo H-susceptibility of N-linked glycans as compared to the control, and a lack of immunoreactivity with an antibody specific for plant complex glycans, indicates that the recombinant protein is targeted to ER-derived protein bodies and contains only high mannose glycans.

Discussion

The targeting of proteins to specific intracellular regions through signals on mRNA molecules is an efficient and common mechanism of plant and animal cells. Sorting of ER-resident proteins from other proteins of the secretory system (e.g. those destined for the cell surface) requires specific targeting information contained either in the structure of these proteins or in the corresponding mRNA molecules. In rice seeds, RNA targeting provides a mechanism to target two different families of storage proteins, both synthesized concurrently in the same endosperm cells during seed development, to specific subdomains of the ER in a way that determines the final subcellular locale of the proteins. The transport of prolamin and glutelin storage proteins along different pathways (viz, to ER-derived protein bodies and to protein storage vacuoles, respectively) is achieved by a differential targeting of their respective mRNAs: prolamin mRNAs target to protein body-ER and glutelin mRNAs are targeted to cisternal ER, allowing subsequent export of the encoded proteins to protein storage vacuoles via the Golgi complex.

Prolamins, the major storage proteins of the maize endosperm are synthesized on the rough ER and accumulate directly in ER-derived protein bodies. Localization of maize zein proteins in ER-derived protein bodies occurs with no carboxyterminal tetrapeptide sequence KDEL or HDEL, indicating that these proteins have an alterative mechanism for ER retention relying on either protein or mRNA signals.

The present invention demonstrates that γ-zein contains mRNA-targeting sequences that ultimately determine protein localization to ER-derived protein bodies Furthermore, the present invention demonstrates the use of regulatory sequences of the γ-zein gene (that encode putative mRNA localization signals and do not specify mature γ-zein protein) to target a recombinant enzyme of interest, for example an enzyme of pharmaceutical importance, to ER-derived protein bodies. In doing so, the recombinant protein accumulates in a stable storage compartment of maize endosperm cells, and avoids transit through the Golgi complex and the consequent maturation of the human enzyme's N-linked glycans.

The present invention demonstrates the existence of mRNA targeting in the endosperm of maize seeds using expression of recombinant human α-L-iduronidase as a non-limiting example. In the presence of putative signals for mRNA targeting (i.e. those provided by the γ-zein gene 5′ and 3′ untranslated- and signal peptide-sequences), α-L-iduronidase contained sufficient information for targeting to ER-derived protein bodies of maize endosperm cells as determined by immunolocalization and subcellular fractionation studies. In the absence of any requirement for mature-protein-coding sequences, it is apparent that this mechanism for protein localization relies on mRNA targeting.

The present invention provides a strategy for the plant-based synthesis of pharmaceutical and other recombinant proteins, in which the resultant protein contains only high mannose N-linked glycans. Maturation of the recombinant proteins' N-linked glycans to complex forms is avoided since there is no transit of the protein through the plant Golgi complex. This control of glycosylation may be important in the utility of any plant-derived therapeutic as the addition of xylose and/or fucose sugar residues has been shown to elicit immunogenic responses in mammals and to greatly reduce the efficacy of plant-derived recombinant proteins for pharmaceutical or other uses (Bardor, M. et al., Glycobiology 13, 427-434; 2003).

The use of mRNA localization signals as a strategy for ER retention of plant-synthesized recombinant proteins is also particularly attractive for downstream processing, since it does not require the fusion of mature-protein-coding sequences onto the recombinant protein.

The present invention demonstrates that a protein of interest, for example, human α-L-iduronidase, accumulates in ER-derived protein bodies, and that the protein, is enzymatically active. This plant-derived iduronidase, containing high mannose glycans, can be further processed in vitro, as required, rendering the recombinant enzyme potentially suitable for use in human enzyme replacement therapies.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Armstrong C L (1994) Regeneration of plants from somatic cell cultures: Applications for in vitro genetic manipulation. In: The Maize Handbook, M. Freeling and V. Walbot, eds. Springer-Verlag, New York, pp. 663-671.
Bagga S, Adams H P, Rodriguez F D, Kemp J D, Sengupta-Gopalan C (1997) Coexpression of the maize delta-zein and beta-zein genes results in stable accumulation of delta-zein in endoplasmic reticulum-derived protein bodies formed by beta-zein. Plant Cell 9: 1683-1696.
Coleman C E, Herman E M, Takasaki K, Larkins B A (1996) The maize gamma-zein sequesters alpha-zein and stabilizes its accumulation in protein bodies of transgenic tobacco endosperm. Plant Cell 8: 2335-2345.
Choi S B, Wang C, Muench D G, Ozawa K, Franceschi V R, Wu Y, Okita T W (2000) Messenger RNA targeting of rice seed storage proteins to specific ER subdomains. Nature 407: 765-767.
Dalta R S S, Bekkaoni F, Hammerlindi J K, Pilate G, Dunstan D I, and Crosby W L (1993) Improved high level constitutive expression in plant using an AMV RNA44 untranslated leader sequence. Plant Sci. 94: 139-149.
Daniell H, Streatfield S J, Wycoff K (2001) Medical molecular fanning: production of antibodies, biopharmaceuticals and edible vaccines in plants. Trends Plant Sci 6: 219-226.
Depicker A, Stachel S, Dhaese P, Zambryski P, Goodman H M (1982) Nopaline synthase: transcript mapping and DNA sequence. J Mol Appl Genet. 1: 561-573.
Dieryck W, Pagnier J, Poyart C, Marden M C, Gruber V, Bournat P, Baudino S, Merot B. (1997) Human haemoglobin from transgenic tobacco. Nature 386: 29-30.
Fang R X, Nagy F, Sivabramaniam S and Chua N H (1989). Multiple cis regulatory elements for maximal expression of the cauliflower mosaic virus 35S promoter in transgenic plants. Plant Cell 1, 141-150.
Herman E M, Larkins B A (1999) Protein storage bodies and vacuoles. Plant Cell 11: 601-614.
Gomez L, Chrispeels M J (1994) Complementation of an Arabidopsis thaliana mutant that lacks complex asparagine-linked glycans with the human cDNA encoding N-acetylglucosaminyltransferase I. Proc Natl Acad Sci USA 91: 1829-1833.
Kermode, A R (1996) Mechanisms of intracellular protein transport and targeting in plant cells. Crit Rev Plant Sci 15: 285-423.
Kornfeld R, Kornfeld S (1985) Assembly of asparagine-linked oligosaccharides. Annu Rev Biochem 54:631-664.
Kornfeld S, Mellman I (1989) The biogenesis of lysosomes. Annu Rev Cell Biol. 5:483-525.
Li X, Franceschi V, Okita T W (1993a) Segregation of storage protein mRNAs on the rough endoplasmic reticulum membranes of rice endosperm cells. Cell 72: 869-879.
Li X, Wu Y, Zhang D Z, Gillikin J W, Boston R S, Franceschi V R, Okita T W (1993b) Rice prolamin protein body biogenesis: A BiP-mediated process. Science 262: 1054-1056.
Lodish H, Berk A, Zipursky S L, Matsudaira P, Baltimore D, Darnell J E (2000) Molecular Cell Biology 4′^{h} hed. W. H. Freeman & Co., New York.
Ma J K, Drake P M, Christou P (2003) The production of recombinant pharmaceutical proteins in plants. Nat Rev Genet 4:794-805.
Ma J K, Hein M B (1995) Immunotherapeutic potential of antibodies produced in plants. Trends Biotechnol 13: 522-527.
Obermeyer G, Gehwolf R, Sebesta W, Hamilton N, Gadermaier G, Ferreira F, Commandeur U, Fischer R, Bentrup F W (2004) Over-expression and production of plant allergens by molecular farming strategies. Methods 32: 235-240.
Rayon C, Cabanes-Macheteau M, Loutelier-Bourhis C, Salliot-Maire I, Lemoine J, Reiter W D, Lerouge P, and Faye L (1999) Characterization of N-glycans from Arabidopsis. Application to a fucose-deficient mutant. Plant Physiol 119: 725-734.
Rings E H H M, Buller H A, Ekker J (1994) Protein sorting versus messenger RNA sorting. Eur J Cell Biol 63: 161-171.
Scott H S, Bunge S, Gal A, Clarke L A, Morris P, Hopwood J J (1995) Molecular genetics of mucopolysaccharidosis, clinical, and biological implications. Human Mutation 6: 288-302.
Songstad D D, Armstrong C L, Petersen W L (1991) AgNO₃ increases Type II callus production from immature embryos of maize inbred B73 and its derivatives. Plant Cell Rep 9: 699-702.
Staehelin L A (1997) The plant ER: A dynamic organelle composed of large number of discrete functional domains. Plant J 11:1151-1165.
St Johnston D (1995) The intracellular localization of messenger RNAs. Cell 81: 161-170.
Thanavala Y, Yang Y F, Lyons P, Mason H S, Arntzen C. (1995) Immunogenicity of transgenic plant-derived hepatitis B surface antigen. Proc Natl Acad Sci USA. 92: 3358-3361.
Twyman R M, Stoger E, Schillberg S, Christou P, Fischer R (2003) Molecular farming in plants: host systems and expression technology. Trends Biotechnol 21:570-578.
von Schaewen A, Sturm A, O'Neill J, Chrispeels M J (1993) Isolation of a mutant Arabidopsis plant that lacks N-acetyl glucosaminyl transferase I and is unable to synthesize Golgi-modified complex N-linked glycans. Plant Physiol 102: 1109-1118.
Zhao K W, Fault K F, Kakkis E D, Neufeld E F (1997) Carbohydrate structures of recombinant human alpha-L-iduronidase secreted by Chinese hamster ovary cells. J Biol Chem 272: 22758-22765.

1. A method of producing a protein of interest in an endoplasmic reticulum (ER)-derived protein body comprising,
i) providing a plant comprising a nucleotide sequence, the nucleotide sequence comprising a first nucleic acid sequence that encodes a prolamin signal peptide, operatively linked to a second nucleic acid sequence that encodes the protein of interest, operatively linked to a third nucleic acid sequence containing the 3′ untranslated region of a gene encoding a prolamin protein, and
ii) expressing the protein of interest in the plant.

2. The method of claim 1, wherein the protein of interest is a glycoprotein.

3. The method of claim 1, wherein the protein of interest is selected from the group consisting of, a hormone peptide, a vaccine, a nutritional supplement, an antibody, an anticoagulant, a growth factor, an enzyme, a lysosomal protein, a therapeutic protein, and an industrial protein.

4. The method of claim 1, wherein the nucleotide sequence comprises a promoter selected from the group consisting of a seed-specific, a tissue specific, and a constitutive promoter.

5. An expression construct comprising the following operatively linked elements:
P-gamma-5′-UTR-gamma-SP—X-gamma-3′-UTR,
where:
P is a seed-specific, tissue specific, or constitutive promoter;
gamma-5′-UTR is the 5′ untranslated region from the maize gamma-zein gene;
gamma-3′-UTR is the 3′ untranslated region from the maize gamma-zein gene;
gamma-SP is a nucleic acid encoding a signal peptide obtained from the maize gamma-zein gene;
X is a nucleotide sequence encoding a protein of interest fused in-frame to the gamma-SP.

6. The expression construct of claim 5, wherein the protein of interest is selected from the group consisting of human alpha-iduronidase, and a lysosomal enzyme.

7. A plant comprising the expression construct of claim 5.

8. A plant tissue, comprising the expression construct of claim 5.

9. A plant seed comprising the expression construct of claim 5.

10. A plant cell comprising the expression construct of claim 5.

11. The plant of claim 7, wherein the plant is a monocot species or a dicot species.

12. The tissue of claim 8, wherein the plant is a monocot species or a dicot species.

13. The seed of claim 9, wherein the plant is a monocot species or a dicot species.

14. The plant cell of claim 10, wherein the plant is a monocot species or a dicot species.
